(19) United States
(12) Plant Patent Application Publication
(10) Pub. No.: US 2013/0019361 P1
(43) Pub. Date: Jan. 17, 2013

(54) Hosta plant named 'Hands Up'

(50) ;

(71) Applicant: Marco Fransen, Tel Aar (NL)

(72) Inventor: Marco Fransen, Tel Aar (NL)

(21) Appl. No.: 13/135,822

(22) Filed: Jul. 14, 2011

(51) Int. Cl. A01H 5/00

(52) U.S. Cl. PLT/353

(57) ABSTRACT

A new and distinct Hosta cultivar named ‘Hands Up’ is disclosed, characterized by a distinctive upright plant habit, large flowers, vigorous plant habit and strong foliage, with good resistance to environmental stressors. The near variety is a Hosta, typically used for outdoor ornamental purposes.

### LATIN NAME OF THE GENUS AND SPECIES

Hosta hybrid

### VARIETY DENOMINATION

‘HANDS UP’

### BACKGROUND OF THE INVENTION

The new cultivar was discovered as an induced mutation using the known chemical colchicine, of Hosta hybrid ‘Praying Hands’ unpatented. ‘Hands Up’ was discovered by the inventor, Marco Fransen, a citizen of the Netherlands in October of 2008, at a research facility in Ter Aar, The Netherlands. The intent of the breeding program was to create new tetraploid Hosta hybrids with stronger foliage and plant vigor.

Directly after discovery of the tetraploid mutation, the inventor initiated cultivation in tissue culture, during October of 2008, in a commercial laboratory in Iribov Heerhugowaard, the Netherlands. Subsequently ‘Hands Up’ has been reproduced by micro-propagation and has shown that the unique features of this cultivar are stable and reproduced true to type through several generations.

### SUMMARY OF THE INVENTION

The cultivar ‘Hands Up’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘Hands Up’ These characteristics in combination distinguish ‘Hands Up’ as a new and distinct Hosta cultivar:

1. Distinctive, upright plant habit.

2. Thick foliage.

3. Large flowers.

4. Vigorous plant.

### COMPARISON TO PARENT

Plants of the new cultivar ‘Hands Up’ are similar to plants of the parent; Hosta hybrid ‘Praying Hands’ in most horticultural characteristics, however, plants of the new cultivar ‘Hands Up’ are more vigorous and produce thicker, stronger leaves than the parent variety. Additionally, the new variety produces larger flowers than ‘Praying Hands’. Leaves of ‘Hands Up’ have a wider variegation on the margin than ‘Praying Hands’.

### COMMERCIAL COMPARISON

The most similar commercial Hosta hybrid variety available to compare to ‘Hands Up’ is the parent variety, as the very strong upright habit is not commonly found in Hostas.

### BRIEF DESCRIPTION OF THE PHOTOGRAPH

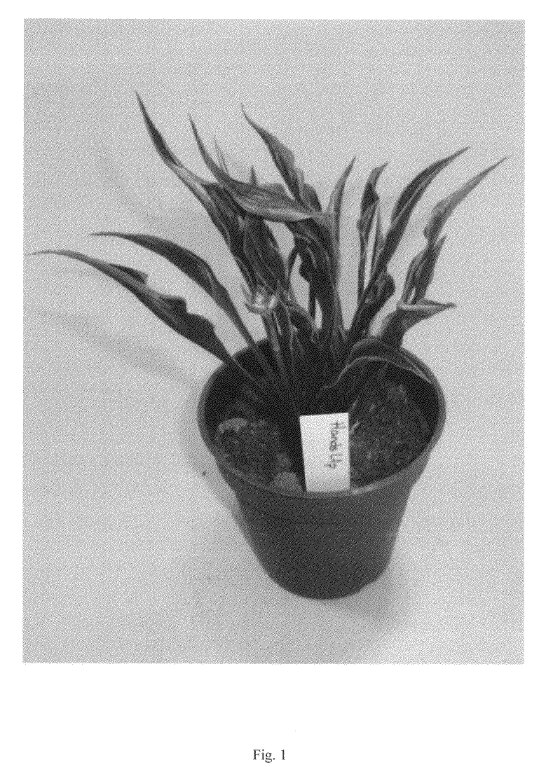

The accompanying photograph in FIG. 1 illustrates in full color a typical plant of ‘Hands Up’ grown outdoors. This plant is approximately 6 months old, shown in a 1 liter pot. The photograph was taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart, 2001, except where general terms of ordinary dictionary significance are used. The following observations and measurements describe ‘Hands Up’ plants grown in a commercial nursery in the Netherlands, from February of 2011 through June of 2011. Temperatures were 5° C. to 20° C. at night and 10° C. to 30° C. during the day. Measurements and numerical values represent averages of typical plant types. No growth regulators or special treatments were used.
Botanical classification: Hosta hybrid ‘Hands Up’
Propagation:
   Time to initiate roots.—Approximately 14 to 21 at 23° C.
   Root description.—Fleshy with moderate side roots.
Plant:
   Growth habit.—Upright, herbaceous perennial plant.
   Height.—Approximately 29 cm in a 1 liter pot.
   Plant spread.—Approximately 20 cm.
   Growth rate.—Moderate.
   Branching characteristics.—Branches emerge directly from the roots.
   Age of plant described.—Approximately 4 months from a stage 3 tissue culture.
Foliage:
   Leaf.—Quantity per plant: Average 50 Leaf Type: Simple, basal emerging Average Leaf Length: Approximately 22 cm Average Leaf Width: Approximately 9.7 cm Apex: Acute Base: Attenuate. Overall Leaf Shape: Lanceolate to lanceolate-oblong Overall Leaf Aspect: Slightly arching, undulating. Margin: Wavy Texture of top surface: Smooth Texture of bottom surface: Smooth Appearance of top surface: Slightly leathery. Appearance of bottom surface: Slightly leathery. Pubescence: No pubescence on leaves Color: Young foliage upper side: Near RHS Green 137A, margin near RHS Yellow-Green 151A Young foliage under side: Near RHS Green 137A, margin near RHS Yellow-Green 151A Mature foliage upper side: Near RHS Green 137A, margin near RHS Yellow-Green 151A Mature foliage under side: Near RHS Green 137A, margin near RHS Yellow-Green 151A Venation: Type: Pinnate. Venation coloration upper side: Near RHS Green 137B Venation coloration under side: Near RHS Green 137B.
   Leaf petiole.—Length: Approximately 8.4 cm Width: Approximately 2.5 cm Color: Upper side: Near RHS Green 137B Under side: Near RHS Green 137B Strength: Very strong.
   Other foliage characteristics.—Thick foliage, resistant to mechanical damage and environmental stressors.
Flower: Plants were not in flower at the time of the initial description.
Other characteristics:
   Disease resistance.—Better resistance to diseases or pests has been observed in this variety, due to the strong, thick foliage.
   Temperature tolerance.—Hardy perennial, tolerates a temperature range from approximately −30° C. to at least 40° C.
   Drought tolerance.—No tolerance for drought.

What is claimed is:

1. A new and distinct cultivar of Hosta plant named ‘Hands Up’ as herein illustrated and described.
